(19) United States
(12) Patent Application Publication
(10) Pub. No.: US 2024/0238195 A1
(43) Pub. Date: Jul. 18, 2024

(54) METHODS AND COMPOSITIONS FOR INHIBITING A NOXIOUS INSULT

(71) Applicants: Oneel Patel, Mooroolbark (AU); Joseph James Ischia, Camberwell (AU); Damien Michael Bolton, Hawthorn (AU); Graham Sherard Baldwin, Pascoe Vale South (AU); Arthur Shulkes, Ascot Vale (AU)

(72) Inventors: Oneel Patel, Mooroolbark (AU); Joseph James Ischia, Camberwell (AU); Damien Michael Bolton, Hawthorn (AU); Graham Sherard Baldwin, Pascoe Vale South (AU); Arthur Shulkes, Ascot Vale (AU)

(21) Appl. No.: 18/616,477

(22) Filed: Mar. 26, 2024

Related U.S. Application Data
(63) Continuation of application No. 17/058,441, filed on Nov. 24, 2020.

(30) Foreign Application Priority Data
May 25, 2018 (AU) 2018901826

(51) Int. Cl. A61K 9/00; A61K 33/30; A61P 9/10

(52) U.S. Cl. CPC A61K 9/0019; A61K 33/30; A61P 9/10

(57) ABSTRACT

The present invention provides a method of inhibiting an effect of a noxious insult, such as an ischaemia-reperfusion injury, to an organ of a subject, the method comprising administering zinc, or a pharmaceutically acceptable salt thereof, such as Zinc Chloride, to a subject in need thereof, wherein the zinc, or the pharmaceutically acceptable salt thereof, is administered to the subject intravascularly in an amount that is sufficient to inhibit the effect of the noxious insult.

### FIELD OF THE INVENTION

The present invention relates generally to methods and compositions for inhibiting an effect of a noxious insult, such as ischaemia and/or reperfusion, to an organ, more specifically to methods and compositions for inhibiting an effect of a noxious insult to an organ such by intravascularly administering zinc, or a pharmaceutically acceptable salt thereof, to a subject in need thereof.

### BACKGROUND OF THE INVENTION

Noxious insults to an organ, whether they be acute (e.g., ischaemia, ischaemia-reperfusion, drug toxicity, shock, stroke, sepsis, trauma, infection, inflammation) or chronic (e.g., hypertension, diabetes, heart failure, lupus, infection, inflammation) in nature, can have a broad spectrum of adverse consequences, ranging from diminished organ function to complete organ failure. Of the different types of noxious insults, ischaemia and ischaemia-reperfusion are regarded as particularly problematic, especially in the context of surgical procedures where temporary vascular obstruction is required to prevent excessive blood loss, or in transplantation where blood flow is temporarily removed.

Ischaemia is generally characterised by insufficient blood supply to an organ as a result of an obstruction, whether by physiological or pathophysiological processes that lead to narrowing of the blood vessels that supply the organ (e.g., arterial narrowing) or during surgical procedures (e.g., arterial clamping). The decrease in blood flow leads to reduced oxygen supply to the organ, triggering a complex series of biological events that impact the structure and/or function of the organ.

In many cases, ischaemia is followed by the resumption of blood flow, also referred to as reperfusion. Ischaemia-reperfusion can lead to the production of excessive amounts of reactive oxygen species (ROS) and reactive nitrogen species (RNS), causing oxidative stress that results in a series of adverse biological events, such as changes in mitochondrial oxidative phosphorylation, depletion of ATP, inflammation, increased intracellular calcium and activation of protein kinases, phosphatases, proteases, lipases and nucleases. These, in turn, lead to loss of cellular function and/or integrity. Independent studies have shown that the inflammatory response induced by ischaemia-reperfusion is largely responsible for tissue and organ damage.

The typical short-term response to ischaemia-reperfusion injury comprises disruption of vascular homeostatic mechanisms, including vasoconstriction, thrombosis, and increased vascular permeability, as well as the activation of inflammatory responses which ultimately lead to fibrosis. The long-term pathophysiological responses to ischaemia vary depending on the organ involved and the severity of the ischaemic event. For example, ischaemia in the brain has been linked to depression, seizure, acute necrosis and delayed neurodegeneration. It is also known that long-lasting ischaemia, wherein occlusion occurs for more than a few minutes, can bring about cell death and permanent organ damage.

Ischaemia-reperfusion injury is also a critical factor in determining the extent of organ damage following hypoxia, stroke, heart attack, and organ transplantation. For example, in organ transplantation, ischaemia-reperfusion injury is a major contributing factor in initiating the cascade of responses that result in episodes of delayed graft function and immunological rejection. These episodes, in turn, are widely believed to be the single most critical factor in the length of graft survival, with a strong correlation observed between the incidence of delayed graft function and graft half-life. While progress has been made in lengthening the survival time of cadaver grafts that are free of acute rejection, there has been little or no progress made in lengthening the survival time of cadaver grafts having episodes of delayed graft function.

Several different approaches have been investigated to treat or prevent the effects of a noxious insult to an organ, such as ischaemia-reperfusion injury. In organ transplantation, for example, these include the administration of agents such as cyclosporine and trimetazidine, muromonab-CD3 (OKT3 monoclonal antibody), mycophenolate mofetil, tacrolimus or induction therapy. However, these strategies have so far failed to improve the survival rates of recipients of cadaver grafts suffering from episodes of delayed graft function.

Other strategies that have been employed to protect organs from the effects of noxious insults, include the delivery of anti-platelet agents (e.g. aspirin, abciximab), anti-coagulants (e.g. warfarin, tissue plasminogen activator (tPA)), anti-inflammatory agents (e.g. aspirin), diuretics (e.g. furosemide), vasodilators (e.g. nitroglycerine, ACE inhibitors), and anti-hypertensive medications (e.g. atenolol). Whilst these drugs reduce the causative factors involved in arterial blockage, they do not appear to provide protection against an ischaemic insult. Moreover, not all patients benefit from such treatments, due in part to factors including drug insensitivity, drug toxicities, drug interactions and other risk factors (e.g. hemorrhage). In addition to drug-based strategies, restoring blood flow by angioplasty, arterial stenting, coronary bypass, and treatment with thrombolytic drugs (e.g. tPA) have also been adopted in an attempt to diminish the adverse effects associated with an ischaemic insult. Whilst these treatments may improve patient prognosis, organ damage can still occur, including the acute risk of vessel rupture and further ischaemic damage, delayed risks arising from restenosis or re-occlusion of the occluded vessel leading to additional ischaemic events.

Finding an effective strategy to treat and/or prevent the effects of a noxious insult to an organ remains a challenge, attributed at least in part to the nature and variability of noxious insults, which involve a complex interplay of regulatory and inflammatory mechanisms. Thus, there remains an urgent need for a method of inhibiting the effect of a noxious insult to an organ.

### SUMMARY OF THE INVENTION

In a first aspect, the present invention provides a method of inhibiting an effect of a noxious insult to an organ of a subject, the method comprising administering zinc, or a pharmaceutically acceptable salt thereof, to a subject in need thereof, wherein the zinc, or the pharmaceutically acceptable salt thereof, is administered to the subject intravascularly in an amount that is sufficient to reduce or inhibit the effect of the noxious insult.

In a second aspect, there is provided zinc, or a pharmaceutically acceptable salt thereof, for use in inhibiting an effect of a noxious insult to an organ of a subject, wherein the zinc, or the pharmaceutically acceptable salt thereof, is administered intravascularly to a subject in need thereof in an amount that is sufficient to inhibit the effect of the noxious insult.

In a third aspect, there is provided use of zinc, or a pharmaceutically acceptable salt thereof, in the manufacture of a medicament for inhibiting an effect of a noxious insult to an organ of a subject, wherein the zinc, or the pharmaceutically acceptable salt thereof, is formulated for intravascular administration to a subject in need thereof in an amount that is sufficient to inhibit the effect of the noxious insult.

### BRIEF DESCRIPTION OF FIGURES

FIG. 1. Serum creatinine levels (μmol/L) following 60 minute ischaemia in sheep who received zinc chloride (ZnCl₂) intravenously at 24 hours and 4 hours prior to ischaemia (n=1-4; ZnCl₂ from 0.1 to 10 mg/kg of body weight). Controls (n=4) received saline intravenously at 24 hours and 4 hours before undergoing a unilateral nephrectomy (*: p<0.05 versus Controls).

FIG. 2. Serum urea levels (mmol/L) following 60 minute ischaemia in sheep who received ZnCl₂ intravenously at 24 hours and 4 hours prior to ischaemia (n=1-4; ZnCl from 0.1 to 10 mg/kg of body weight). Controls (n=4) received saline intravenously at 24 hours and 4 hours before undergoing a unilateral nephrectomy (*: p<0.05 versus Controls).

FIG. 3. Area Under the Curve (AUC) for serum creatinine and serum urea levels in sheep who received ZnCl₂ (10 mg/kg of body weight) intravenously at 24 hours and 4 hours prior to ischaemia (**: p<0.05 versus Controls).

FIG. 4. Serum creatinine and urea levels following 60 minute ischaemia in sheep who received ZnCl₂ (10 mg/kg of body weight) or the flavonoid based antioxidant intravenously at 24 hours and 4 hours prior to ischaemia. Controls (n=4) received saline intravenously at 24 hours and 4 hours before undergoing a unilateral nephrectomy.

FIG. 5 shows the perfusion of ZnCl₂ (50 μM) into a human kidney via the segmental renal artery (A) and the renal tissue that was subsequently removed for analysis (B). The blood supply to the excised tissue was provided by the renal artery into which the ZnCl₂ solution was administered.

FIG. 6. Hypoxia inducible factor 1-alpha (HIF-1α) protein expression in tissue derived from ZnCl₂-perfused human kidneys (n=2). Protein expression was measured by Western Blot. The results are shown as a percentage (%) of HIF-1α protein expression in saline-perfused human kidneys (n=2).

FIG. 7. HIF-1α protein expression in immortalised human kidney tubular renal HK-2 cells treated with ZnCl₂ (n=3; 50 μM) over 24 hours. Samples were collected and HIF-1α protein expression was determined by Western Blot. The level of expression is shown relative to the expression of the house-keeping gene, glyceraldehyde 3-phosphate dehydrogenase (GAPDH); *; p<0.05.

FIG. 8. (A) HIF-1α, HIF-2α, HIF-3α, pAKT and pMAPK protein expression in human renal carcinoma cells (ACHN) exposed to cobalt chloride (CoCl₂; 150 μM) or zinc chloride (ZnCl₂; 50 μM). The results show an increase in HIF-1α and HIF-2× protein expression in human renal carcinoma cells at 4 hours following exposure to ZnCl₂ when compared to controls, as determined by Western Blot. (B) Quantitation of the protein expression data shown in FIG. 8(A). (C) HIF-1α, HIF-2α, HIF-3α, pAKT and pMAPK protein expression in normal human proximal tubular HK-2 cells exposed to cobalt chloride (CoCl₂; 150 μM) or zinc chloride (ZnCl₂; 50 μM). The results show an increase in HIF-1α and HIF-2α protein expression in human renal carcinoma cells at 4 hours following exposure to ZnCl₂ when compared to controls, as determined by Western Blot. (D) Quantitation of the protein expression data shown in FIG. 8(C).

FIG. 9. Serum creatinine and urea levels following renal ischaemia-reperfusion injury in sheep. Sheep were subjected to uninephrectomy and 60 min of renal ischaemia followed by reperfusion. Serum creatinine (A; μmol/L) and serum urea (B; mmol/L) were measured before Zn treatment (baseline), before ischaemia (Day 0) and for 7 days after reperfusion. Sheep were preconditioned with a single dose of 0.5 mg/kg ZnCl₂ either at 24 hr or 4 hr only, or with double dose at 24 and 4 hr prior to 60 min ischaemia. Data are expressed as mean±SEM values (n=4, except 24 hr only group, n=1). 0.5 mg/kg ZnCl₂ administered as two doses one at 24 and the other at 4 hr prior to ischaemia significantly (p<0.05) reduced the creatinine rise over time compared to single dose given only at either 24 hr or 4 hr prior to ischaemia.

FIG. 10. Serum creatinine and urea levels following renal ischaemia-reperfusion injury in sheep. Sheep were subjected to uninephrectomy and 60 min of renal ischaemia followed by reperfusion. Sheep were preconditioned with a single dose of 0.5 mg/kg ZnCl₂ either at 24 hr or 4 hr only, or with double dose at 24 and 4 hr prior to 60 min ischaemia. Data are expressed as mean±SEM values (n=4, except 24 hr only group, n=1). 0.5 mg/kg ZnCl₂ administered as two doses one at 24 and the other at 4 hr prior to ischaemia significantly (p<0.05) reduced the ischaemic burden (area under curve) expressed either creatinine (A) or urea (B) rise over time compared to single dose given only at either 24 hr or 4 hr prior to ischaemia.

FIG. 11. Cross-section of sheep left ventricle rings following prolonged myocardial ischaemia-reperfusion injury. Representative digital photos (triphenyltetrazolium chloride (TTC) staining, B) relative to the area of myocardium at risk (pale, non-Evans blue stained, A). Absence of white areas when compared to untreated sheep is indicative of protection against myocardial ischaemia reperfusion injury by zinc preconditioning.

### DETAILED DESCRIPTION OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

All publications mentioned in this specification are herein incorporated by reference in their entirety.

It must be noted that, as used in the subject specification, the singular forms “a”, “an” and “the” include plural aspects unless the context clearly dictates otherwise. Thus, for example, reference to “an agent” includes a single agent, as well as two or more agents: reference to “the composition” includes a single composition, as well as two or more compositions; and so forth.

The present invention is predicated on the surprising finding that the intravascular administration of zinc is capable of protecting an organ, such as the kidney and heart, against the adverse effects of a noxious insult such as ischaemia-reperfusion injury. Accordingly, in a first aspect the present invention provides a method of inhibiting an effect of a noxious insult to an organ of a subject, the method comprising administering zinc, or a pharmaceutically acceptable salt thereof, to a subject in need thereof, wherein the zinc, or the pharmaceutically acceptable salt thereof, is administered to the subject intravascularly in an amount that is sufficient to inhibit the effect of the noxious insult.

### Noxious Insult

The term “noxious insult”, as used herein, means an insult that is harmful or injurious to an organ. It will be understood that the effect of the noxious insult may manifest itself in a number of ways, depending on the nature of the insult. For example, the effect of a noxious insult may manifest itself as a structural change to the organ (e.g., an anatomical and/or histological change), illustrative examples of which include the appearance of an inflammatory cell infiltrate, cellular hypertrophy, cell death (e.g., necrosis, programmed cell death) and extracellular matrix deposition (i.e., scarring). In an embodiment, the effect of the noxious insult will manifest itself as programmed cell death (apoptosis) or cell necrosis. Apoptosis, or programmed cell death, is a physiological mechanism for removing senescent, damaged or abnormal cells. Apoptosis is typically initiated by an endonuclease and is characterized by DNA fragmentation into multiples of 180-200 base pairs. Apoptotic cells are ingested by macrophages or neighboring cells, typically without release of proteolytic enzymes or toxic oxygen species, and in the absence of inflammation. By contrast, necrosis is a pathological process that affects populations of cells and results in focal tissue destruction, inflammation and often serious systemic consequences.

Alternatively, or in addition, the effect of a noxious insult may manifest itself as a change to the function of the organ. Persons skilled in the art will understand that the nature and/or extent of the functional change will depend on the organ affected and the nature and/or severity of the noxious insult. For instance, a noxious insult to a kidney may manifest itself as an increase in serum creatinine, an increase in serum urea, changes to creatinine clearance, or combinations thereof. Methods of determining a change in organ function will be familiar to persons skilled in the art. In another embodiment, the effect of a noxious insult to an organ can be determined by changes to the expression of biological markers known to be associated with noxious injury. Illustrative examples are neutrophil gelatinase-associated lipocalin (NGAL), kidney injury molecule-1 (KIM-1), and N-acetyl-glucosaminidase (NAG) which are increased following an ischaemic insult.

Noxious insults will be familiar to persons skilled in the art, illustrative examples of which include ischaemia, ischaemia-reperfusion injury and contrast media-induced injury (e.g., resulting in contrast-induced nephropathy). In an embodiment disclosed herein, the noxious insult is ischaemia. As noted elsewhere herein, ischaemia is a term used to describe an insufficient supply of blood to an organ or tissue, typically as a result of vascular obstruction, whether by physiological or pathophysiological processes that lead to narrowing of the blood vessels that supply the organ or tissue (e.g., arterial narrowing) or during a surgical procedure (e.g., arterial clamping, or hypoperfusion with bleeding or cardiac insufficiency). The reduction in blood flow leads to reduced oxygen supply to the organ or tissue, triggering a complex series of biological events that impact the structure and/or function of the organ or tissue. Ischaemia can result from any of a number of causative events, illustrative examples of which include hypoxia, stroke, organ failure (e.g., heart attack, kidney failure) and organ transplantation.

In some instances, ischaemia is followed by the resumption of blood flow, or “reperfusion”. The combination of an initial ischaemic insult and subsequent reperfusion is often referred to as “ischaemia-reperfusion”. Without being bound by theory or by a particular mode of application, injury to an organ or tissue arising from ischaemia-reperfusion is attributed, at least in part, to the production of excessive amounts of reactive oxygen species (ROS) and reactive nitrogen species (RNS), causing oxidative stress that results in a series of adverse biological events, such as changes in mitochondrial oxidative phosphorylation, depletion of ATP, increased intracellular calcium and activation of protein kinases, phosphatases, proteases, lipases and nucleases that lead to loss of cellular function and/or integrity. In an embodiment disclosed herein, the noxious insult is ischaemia-reperfusion.

Ischaemia-reperfusion injury may result in microvascular dysfunction that is manifested, for example, as impaired endothelium-dependent dilation of arterioles, enhanced fluid filtration, leukocyte accumulation in vascular capillaries and plasma protein extravasation in postcapillary venules. Independent studies have found that activated endothelial cells in the microcirculation produce more oxygen radicals, but less nitric oxide, during the initial period following reperfusion. The imbalance between superoxide and nitric oxide in endothelial cells can lead to the production and release of inflammatory mediators (e.g. platelet-activating factors, tumor necrosis factor) and the enhanced biosynthesis of adhesion molecules that mediate leukocyte-endothelial cell adhesion. The inflammatory mediators released as a consequence of reperfusion also appear to activate endothelial cells in remote organs that are not exposed to the initial ischaemic insult. This distant response to ischaemia-reperfusion injury can result in leukocyte-dependent microvascular injury that is characteristic of multiple organ dysfunction.

The term “ischaemic” is used to describe an organ, a cell, a tissue or a sample that is affected to some extent by ischaemia or ischaemia-reperfusion.

In an embodiment disclosed herein, the noxious insult is warm ischaemia. Warm ischaemia is a term typically used to describe an obstruction of blood flow to an organ at physiological temperatures (e.g., around 36.5° C.). In another embodiment, the noxious insult is cold ischaemia. Cold ischaemia is a term typically used to describe an ischaemic event when the temperature of the organ or tissue has been reduced (e.g., chilled) to below physiological temperatures. This can occur while the tissue or organ is still in the body or after it is removed from the body, for example, if the organ or tissue is to be transplanted to another subject. Reducing the temperature of an organ or tissue, also referred to as hypothermic preservation, is based on the principle that cooling an organ or tissue will inhibit the enzymatic processes within the organ and, hence, reduce the extent of cellular damage in the presence of reduced blood supply. Previous studies have indicated a 2-3 fold decrease in metabolism for every 10° C. reduction in temperature. This slows the depletion of adenosine triphosphate (ATP) and also inhibits the degrading processes (phospholipid hydrolysis). However, under hypothermic conditions, the metabolic rate still remains at about 10% and, therefore, over time, the hypoxic conditions can still cause substantial injury (cold ischaemia injury).

It is to be noted that any organ exposed to a noxious insult may benefit from the methods described herein, illustrative examples of which include kidney, heart, liver and brain. In an embodiment, the organ of the subject is selected from the group consisting of kidney, liver, heart and brain. In a further embodiment, the organ of the subject is selected from the group consisting of kidney and heart. In yet a further embodiment, the organ of the subject is a kidney. In yet a further embodiment, the organ of the subject is the heart.

In an embodiment disclosed herein, the noxious insult is renal ischaemia (i.e., an ischaemic insult to a kidney) or renal ischaemia-reperfusion.

In another embodiment disclosed herein, the noxious insult results from a surgical procedure. Examples of surgical procedures that would place an organ at risk of ischaemic injury would be known to persons skilled in the art. In some instances, the surgical procedure requires active occlusion of the vasculature supplying blood to the organ or tissue so as to minimize or otherwise avoid the loss of blood during the procedure. The type of surgical procedure requiring active vascular occlusion will be known to persons skilled in the art, illustrative examples of which include tumour resection during partial nephrectomy and organ harvesting for transplantation. In an embodiment disclosed herein, the ischaemia arises during resection of a renal tumour (e.g., renal cell carcinoma). For instance, resection of a renal tumour typically requires a partial nephrectomy via open or laparoscopic surgery, where the renal hilar is clamped to prevent excessive bleeding and allow sufficient time for precise tumour excision, pelvicalyceal suture repair and for securing parenchymal haemostasis. In an embodiment disclosed herein, the noxious insult is to a donor kidney during a kidney transplant. This can occur, for example, when blood supply to the donor kidney is obstructed, interrupted or otherwise restricted, such as cessation of blood flow at death in a cadaver donor, occlusion of blood supply to the donor kidney in situ (e.g., via clamping of renal arteries) prior to the removal of the donor kidney from the donor (living or cadaver) for subsequent transplantation, cessation of blood flow to the donor kidney following its removal from the donor, or combinations thereof.

In another embodiment, the noxious insult is contrast media-induced toxicity. Contrast media have been shown to act on distinct anatomic sites within the kidney and exert adverse effects via multiple mechanisms, including a direct cytotoxic effect on the renal proximal tubular cells, enhanced cellular damage by reactive oxygen species, and increased resistance to renal blood flow. They have also been shown to exacerbate renal vasoconstriction, particularly in the deeper portions of the outer medulla, which can worsen pre-existing abnormal vascular pathobiology.

As used herein in connection with an effect of a noxious insult, the term “inhibit” refers to any decrease, diminution, attenuation, reduction or reversal of damage to an organ of a subject that is attributable, at least in part, to the noxious insult, whether any such inhibition is transient, partial or complete. The extent of damage to an organ that is attributable, at least in part, to the noxious insult may be determined by methods known to persons skilled in the art. Such methods are likely to depend on the type of organ to be assessed. As an illustrative example, damage to a kidney as a result of a noxious insult such as ischemia-reperfusion may be assessed by changes to kidney function, which may manifest as changes to the concentration of serum creatinine and/or serum urea in the subject. In another illustrative example, damage to brain tissue in a subject may be assessed by changes to cognitive function. In yet another illustrative example, damage to an organ as a result of a noxious insult may be assessed by histological changes to the organ, such as the presence of tissue necrosis and/or cell apoptosis. Methods for assessing histological changes to an organ will be familiar to persons skilled in the art, illustrative examples of which include immunocytochemistry (e.g., staining for markers of cell apoptosis) and the use of histological stains (e.g., Evan's blue, Haematoxylin and Eosin) as described elsewhere herein.

### Zinc

As noted elsewhere herein, the inventors have surprisingly found that the intravascular administration of zinc to a subject protects the subject's organs, such as the subject's kidneys, from the adverse effects of a noxious insult, such as ischaemia or ischaemia-reperfusion. It is to be understood that any form of zinc, or a pharmaceutically acceptable salt thereof, can be administered to the subject in accordance with the present invention, as long as the form is suitable for delivering the zinc, or the pharmaceutically acceptable salt thereof, to the organ in need.

Suitable forms of zinc, or pharmaceutically acceptable salts thereof, would be known to persons skilled in the art. Illustrative examples include organic and inorganic salts of zinc, such as zinc salts derived from mineral acids (hydrochloric acid, sulphuric acid, phosphoric acid, etc), organic acids (e.g., acetic acid, malic acid, fumaric acid, etc) an combination salts (e.g., carbonate/hydroxide salts, ammonium salts, chelates, etc). The methods disclosed herein also contemplate various combinations of zinc, including organic and inorganic salts thereof.

The term “inorganic zinc”, as used herein, refers to the divalent cation Zn²⁺ and inorganic salts thereof. Suitable forms of inorganic zinc will be known to persons skilled in the art, illustrative examples of which include zinc chloride (ZnCl₂), tetrabasic zinc chloride (ZnsCl₂(OH)₈), zinc oxide (ZnO), zinc sulfate (ZnSO₄), and tetrabasic zinc chloride (ZnsCl₂(OH)₈). The percentage of elemental zinc may vary in such forms. For example, approximately 23% of zinc sulfate comprises elemental zinc. Thus, 220 mg of zinc sulfate will contain about 50 mg of elemental zinc.

The term “organic zinc”, as used herein, refers to organic zinc complexes as well as zinc proteinates, zinc chelates and salts with organic molecules and compounds, and zinc amino acid complexes. Suitable forms of organic zinc will be known to persons skilled in the art, illustrative examples of which include zinc histidine, zinc methionine (ZnMet), Zinc lysine (ZnLys) complexes, zinc acetate (Zn(O₂CCH₃)₂), zinc ascorbate (C₁₂H₁₄ZnO₁₂), zinc aspartate (C₈H₁₀N₂O₈Zn₂H), zinc butyrate (Zn(C₄H₇O₂)), zinc carbonate (ZnCO₃), zinc citrate (Zn₃(C₆H₅O₇)₂), zinc gluconate (Zn(Cl₂H₂₂O₁₄)), zinc glycinate (C₄H₈N₂O₄Zn), zinc histidinate (C₁₂H₁₆N₆₀₄Zn), zinc ketoglutarate (C₅H₄O₅Zn), zinc lactate (Zn(C₃H_{S}O₃)₂), zinc malate (C₄H₄O₅Zn), zinc picolinate (C₁₂H₈N₂O₄Zn), zinc propanoate (C₆H₁₀O₄Zn), zinc stearate (C₃₆H₇₀O₄Zn), and zinc succinate (C₄H₄O₄Zn). As with inorganic zinc salts, the percentage of elemental zinc may vary in these forms. For example, with zinc arginate, 300 mg will provide about 30 mg of elemental zinc.

In an embodiment disclosed herein, the zinc is administered in the form of zinc chloride.

In one embodiment, the zinc, or pharmaceutically acceptable salt thereof, is administered to the subject as a suitable solution. The solution may be formed from the zinc, or pharmaceutically acceptable salt thereof, and a standard liquid carrier used for typical intravascular administration, such as a saline solution or plasma.

Preferably, the zinc, or a pharmaceutically acceptable salt thereof, is administered in a form that allows the administered dose to be controlled so as to provide an amount of zinc that is sufficient to inhibit the effect of the noxious insult, also referred to herein as a “therapeutically effective amount”, whilst avoiding any adverse effects typically associated with excessive amounts of zinc (i.e., zinc toxicity).

The National Academy of Sciences (NAS) estimates a recommended dietary allowance (RDA) for zinc of 11 mg/day for males, which is the equivalent of 0.16 mg/kg of body weight per day for the average adult male (70 kg). For females, the RDA has been estimated as 8 mg/day, or 0.13 mg/kg of body weight for an average adult female (60 kg). Lower zinc intake has been recommended for infants (2-3 mg/day) and children (5-9 mg/day). Whilst the RDA aims to provide a level of adequate nutritional status for most of the population, extra dietary levels of zinc are recommended for women during pregnancy and lactation: an RDA of 11-12 mg/day has been set for pregnant women and 12-13 mg/day for lactating women.

It is to be noted that dietary (oral) administration of zinc is subject to large variations in absorption, distribution, metabolism and clearance. In plasma, for example, two-thirds of the zinc is bound to albumin, which represents the metabolically active pool of zinc. This pool of plasma zinc is frequently referred to as loosely bound zinc because albumin has the ability to give up bound zinc to tissues. Zinc excretion is largely facilitated by the pancreas and into the faeces (75%), which contain unabsorbed zinc, as well as biliary and pancreatic secretions. The other 25% of zinc is eliminated by renal clearance (i.e., through the urine). Faecal excretion of zinc has been shown to increase as the intake of zinc increases, whereas renal clearance of zinc is relatively stable in subjects with normal kidney function (renal clearance can increase 10-fold in patients with reduced renal function). Minor routes of elimination are saliva secretion, hair loss, and sweat. Since zinc is not stored, the balance between absorption and clearance (homeostasis) is essential to the maintenance of a broad spectrum of zinc-dependent functions. Thus, existing dietary intake of zinc cannot provide adequate control over the therapeutically effective amount of zinc that is required to inhibit the effect of a noxious insult to the kidney.

The inadequate control over the therapeutically effective amount of zinc by dietary intake also means that there is an unacceptable risk of zinc toxicity resulting from an accumulation of zinc that is higher than the RDA. In adults, for example, supplemental doses of zinc of 25-35 mg/day appear to be well tolerated, although intakes as high as 50 mg zinc/day appear to affect copper metabolism. The Australian National Health and Medical Research Council (NHMRC) has set the upper limit of the RDA for zinc at 40 mg/day. The US Environmental Protection Agency (US EPA) has set an oral reference dose (RfD) of 0.3 mg/kg/day for zinc, based on reported lowest observed adverse effect level (LOAEL) from a clinical study of the effects of oral zinc supplementation on copper and iron status. The RfD, which is an estimate of the daily oral exposure to the human population (including susceptible subgroups) that is likely to be without an appreciable risk of adverse health effects over a lifetime, corresponds to 21 mg zinc for a 70 kg male and 18 mg for a 60 kg female and is higher than the recommended acceptable daily intake recommended by the WHO in some instances. On the other hand, the US Food and Nutrition Board has set the tolerable upper intake level (UL) at 40 mg/day for adults older than 19 years. The UL is another form of toxicity risk assessment value designed to protect 97-98% of the population.

As outlined above, there are apparent conflicts between the RDA, RfD and UL values, which primarily reflect the large uncertainties in the ability to associate zinc status with normal states of human health and in detecting mild to moderate zinc deficiency and toxicity endpoints. A comparison of the RDA with RfD nevertheless points to the fact that there is little margin between a safe and unsafe dose of zinc. Unsafe doses of zinc can lead to acute or chronic zinc toxicity. The symptoms of acute zinc toxicity include abdominal pain, nausea and vomiting. Other reported effects include lethargy, anaemia and dizziness. In humans, the acute toxic effects of dietary zinc have been shown to occur at doses of approximately 200 mg or more. ESOD (erythrocyte superoxide dismutase) activity, a measure of copper status, was also shown to decrease following supplementation with 50 mg/day or more for 12 days. Dietary zinc supplements of 50 mg/day for up to 10 weeks have resulted in reductions in haematocrit and serum ferritin. Doses greater than 100 mg/day have resulted in altered ratios of HDL:LDL cholesterol. Copper deficiency and sideroblastic anaemia, associated with chronic zinc ingestion, were reported in an individual who had taken non-prescribed zinc supplements of 26.6-40 mg/day for at least 2 years.

Zinc affects iron and copper uptake at supplemental doses of 50 mg/day and above. Thus, prolonged use of high doses of zinc (chronic zinc toxicity) can result in secondary deficiency of copper, symptoms of which include hypocupraemia, impaired iron mobilisation, anaemia, leukopenia, neutropenia, decreased superoxide dismutase (SOD) (particularly erythrocyte SOD (ESOD)), decreased ceruloplasmin, decreased cytochrome C oxidase, increased plasma cholesterol, increased LDL:HDL cholesterol, decreased glucose clearance, decreased methionine and leucine enkephalins, abnormal cardiac function and impairment of pancreatic enzymes, amylase and lipase. It has also been suggested that excess zinc is atherogenic.

Other symptoms of zinc toxicity include nephrotoxicity (e.g., microscopic hematuria unaccompanied by renal failure and mild albuminuria: regressive kidney lesions, severe diffuse nephrosis, epithelial cell damage in the glomerulus and proximal convoluted tubules, elevated plasma creatinine and urea levels, renal tubular dilation with proteinaceous casts and hemosiderin deposits), neurotoxicity (e.g., lethargy, lightheadedness, staggering, difficulty in writing clearly, anxiety, depression, somnolence, comatose, neuronal and glial cell death), hepatotoxicity (e.g., transiently increased liver enzyme activities, associated with severe gastrointestinal corrosion), cardiovascular toxicity (e.g., premature atrial beats, hypertension secondary to intravascular volume, hypovolemic shock (pulse over 120 beats per minute) and hypertension), carcinogenicity and genotoxicity. Earlier reports show that zinc overdose resulting from oral ingestion of zinc sulfate in large amounts (≥30,000 mg) can result in death. Smaller doses of about 2,000-4,000 mg/day of zinc gluconate has resulted in anemia (consistent with zinc-induced copper deficiency), severe nephrosis, nausea, vomiting, dehydration, electrolyte imbalances, dizziness, lethargy and incoordination, epigastric pain and abdominal pain.

The LD₅₀ values of several zinc compounds (ranging from 186 to 623 mg zinc/kg of body weight per day) have been determined in rats and mice. In rats, zinc acetate was the most lethal compound tested: zinc nitrate, zinc chloride, and zinc sulfate (in order of decreasing toxicity) were less lethal. In mice, the most lethal compound was zinc acetate followed by zinc nitrate, zinc sulfate, and zinc chloride. Ingestion of 390 mg zinc/kg of body weight per day as zinc oxide in the diet for 3-13 days was lethal to 3 of 3 ferrets. An equivalent dose in humans would be approximately 27,000 mg/day. Mortality has also been observed in rats ingesting 191 mg zinc/kg/day as zinc acetate in drinking water for 3 months.

As outlined above, current dietary intake of zinc suffers from inadequate control over the therapeutically available amount, attributed at least in part to differences in absorption, metabolism and clearance, the rates of which are likely to vary from one individual to another. Subcutaneous administration of zinc has also been described, primarily in animal models. However, whilst subcutaneous administration provides some control over the therapeutically available amount of zinc as compared to oral administration, it too suffers from inadequate control over the therapeutically available amount of zinc, as it is generally difficult to account for differences in the rate of absorption from the subcutaneous depot. The inadequate control over the therapeutically available amount of zinc by dietary (oral) or subcutaneous administration introduces unacceptable risks to a subject that are associated with insufficient amounts of zinc (i.e., a lack of a therapeutically effective amount) or excessive amount of zinc (i.e., zinc toxicity).

The present inventors have shown that zinc administered intravascularly (e.g., intravenously and/or intra-arterially) to the organ of concern provides for more precise control over the therapeutically effective amount of zinc (i.e., for inhibiting the effect of the noxious insult) and can therefore be used to avoid or minimize at least some of the adverse effects of zinc toxicity. Moreover, it is shown herein for the first time that, in some instances, higher doses of zinc fail to protect the kidney against the effects of a noxious insult and, in other instances, may exacerbate the adverse effects of a noxious insult. Hence, in preferred embodiments, the amount of zinc administered to the subject in accordance with the methods disclosed herein is preferably controlled so as to provide an amount that is effective to inhibit the effect of the noxious insult (i.e., a therapeutically effective amount) whilst avoiding an adverse effect associated with an excessive amount of zinc.

The zinc, or the pharmaceutically acceptable salt thereof, may be administered intravascularly by any means known to persons skilled in the art. Suitable modes of intravascular administration will be known to persons skilled in the art, illustrative examples of which include intravenous and/or intra-arterial administration. In an embodiment, the method comprises administering the zinc, or the pharmaceutically acceptable salt thereof, to a subject intravenously. In another embodiment, the method comprises administering the zinc, or the pharmaceutically acceptable salt thereof, to a subject intra-arterially. In an embodiment disclosed herein, the zinc, or the pharmaceutically acceptable salt thereof, is administered intravascularly by infusion, such as by direct infusion (e.g., perfusion) into the vasculature of the organ of interest, such as the kidney vasculature (e.g., renal artery). In another embodiment, the zinc, or the pharmaceutically acceptable salt thereof, is administered intravascularly by indirect administration, for example, via a peripheral or central vein. Administration may be performed either laparoscopically or during open surgery and prior to the ischaemic insult. In an embodiment disclosed herein, the zinc, or the pharmaceutically acceptable salt thereof, is administered via perfusion to the organ. In another embodiment disclosed herein, the zinc, or the pharmaceutically acceptable salt thereof, is administered via a peripheral and/or central vein.

The therapeutically effective amount of zinc will vary, depending on factors such as the subject's age, weight and health status (e.g., a pre-existing disease or condition such as acute or chronic renal impairment, diabetes, and hypertension) that may affect zinc bioavailability, having regard, for example, to rates of absorption, metabolism and clearance.

It has been found that zinc administered intravascularly in an amount that is greater than or equal to 1 mg/kg of body weight does not appear to inhibit the adverse effect of an ischaemia-reperfusion insult to kidneys. For instance, zinc administered in an amount that is greater than or equal to 1 mg/kg of body weight did not improve renal function following ischaemia-reperfusion injury, as determined by serum creatinine and serum urea levels. Moreover, zinc administered in even higher amounts (of 2.5 mg/kg of body weight or 10 mg/kg of body weight) worsened renal function following ischaemia-reperfusion injury. Thus, the inventors have surprisingly found that, in some instances, there may be an upper limit to the range of zinc that can be administered intravascularly (e.g., via perfusion) that will provide a therapeutically effective amount to inhibit an effect of a noxious insult to a kidney.

In an embodiment disclosed herein, the zinc, or the pharmaceutically acceptable salt thereof, is administered intravascularly in an amount that is less than 1.0 mg/kg body weight. In another embodiment disclosed herein, the zinc, or the pharmaceutically acceptable salt thereof, is administered intravascularly in an amount that is selected from the group consisting of from about 0.1 mg/kg body weight to about 0.9 mg/kg body weight, preferably from about 0.1 mg/kg body weight to about 0.8 mg/kg body weight, preferably from about 0.1 mg/kg body weight to about 0.7 mg/kg body weight, preferably from about 0.2 mg/kg body weight to about 0.9 mg/kg body weight, preferably from about 0.2 mg/kg body weight to about 0.8 mg/kg body weight, preferably from about 0.2 mg/kg body weight to about 0.7 mg/kg body weight, preferably from about 0.3 mg/kg body weight to about 0.9 mg/kg body weight, preferably from about 0.3 mg/kg body weight to about 0.8 mg/kg body weight, or preferably from about 0.3 mg/kg body weight to about 0.7 mg/kg body weight. In another embodiment disclosed herein, the zinc, or the pharmaceutically acceptable salt thereof, is administered intravascularly in an amount that is about 0.5 mg/kg body weight.

It is to be understood that the amount of zinc, or a pharmaceutically acceptable salt thereof, that represents an upper limit of the dosage range that provides a therapeutically effective amount of zinc in a subject, as herein described, is not definitive, but is likely to be dependent on factors such as, but not limited to, the age, weight, the general health status of the subject and the organ for which protection against a noxious insult is desired. The upper limit, above which the zinc, or the pharmaceutically acceptable salt thereof, will provide little or no therapeutic effect (or may worsen the effect of the noxious insult), can be an approximation, readily determined by persons skilled in the art. For example, a pilot study can be performed by administering a range of doses to a cohort of healthy subjects (controls) and to a cohort of subjects who will undergo surgery on their kidney(s) where vascular occlusion is required. Analyses can then be performed to ascertain the dose at which there is little or no inhibition to the effect of the noxious insult (or a worsening of the effect of the noxious insult) arising from vascular occlusion and/or subsequent reperfusion. This can be ascertained by measuring renal function or other parameters affected by the noxious insult (e.g., number of apoptotic cells: or expression of factors such as hypoxia inducible factor 1-alpha, neutrophil gelatinase-associated lipocalin (NGAL), kidney injury molecule-1 (KIM-1), N-acetyl-glucosaminidase (NAG), NephroCheck (which detects the presence of insulin-like growth-factor binding protein 7, IGFBP7) and tissue inhibitor of metalloproteinases (e.g., TIMP-2) in the urine (which are associated with acute kidney injury).

As noted elsewhere herein, the present inventors have shown that intravascularly administered zinc protects organs, such as kidneys and heart, from the adverse effects of a subsequent noxious insult, such as ischaemia-reperfusion. Thus, it will be understood by persons skilled in the art that the zinc, or the pharmaceutically acceptable salt thereof, is to be administered prior to the noxious insult, whether the noxious insult is anticipated or not. For example, in some instances, a noxious insult is anticipated where the subject is to undergo surgery on a kidney, such as where clamping of a renal artery is required to prevent excessive bleeding. In that instance, the zinc, or the pharmaceutically acceptable salt thereof, is administered prior to the ischaemic insult arising from clamping of the renal vasculature. Thus, in an embodiment, the method comprises administering zinc, or a pharmaceutically acceptable salt thereof, to a subject in need thereof, wherein the zinc, or the pharmaceutically acceptable salt thereof, is administered to the subject intravascularly in an amount that is sufficient to inhibit the effect of the noxious insult and wherein the zinc, or the pharmaceutically acceptable salt thereof, is administered to the subject prior to the noxious insult. The period of time between administering the zinc, or the pharmaceutically acceptable salt thereof, to the subject and the onset of the noxious insult will vary, having regard to factors such as the concentration of zinc to be administered and the age, weight and health of the subject. The period of time is to be chosen to ensure that a therapeutically effective amount of zinc is still present at the onset of the noxious insult to inhibit an adverse effect of the noxious insult, as herein described. The period of time can be determined by persons skilled in the art. In some embodiments, the zinc, or the pharmaceutically acceptable salt thereof, is administered to the subject within 36 hours prior to the noxious insult, preferably within 1 hour, preferably within 2 hours, preferably within 3 hours, preferably within 4 hours, preferably within 5 hours, preferably within 6 hours, preferably within 7 hours, preferably within 8 hours, preferably within 9 hours, preferably within 10 hours, preferably within 11 hours, preferably within 12 hours, preferably within 13 hours, preferably within 14 hours, preferably within 15 hours, preferably within 16 hours, preferably within 17 hours, preferably within 18 hours, preferably within 19 hours, preferably within 20 hours, preferably within 21 hours, preferably within 22 hours, preferably within 23 hours, preferably within 24 hours or preferably 24 hours prior to the noxious insult. In another embodiment disclosed herein, the zinc, or the pharmaceutically acceptable salt thereof, is administered to the subject within 4 hours prior to the noxious insult.

In some instances, it will be understood that multiple doses will be required to achieve a therapeutically effective amount of zinc, or a pharmaceutically acceptable salt thereof. The number of doses can readily be determined by persons skilled in the art, for example, by making an observation as to whether the doses are adequate to inhibit an adverse effect of the noxious insult, as herein described. It will be understood that, where multiple doses are required or desired, the number of doses will depend on factors such as the concentration of zinc, or a pharmaceutically acceptable salt thereof, in the formulation to be administered. For example, for relatively small concentrations of zinc, a greater number of doses are likely to be required to, as compared to a formulation comprising a larger concentration of zinc, where a single dose may suffice to achieve a therapeutically effective amount of zinc in a subject in need thereof. The period of time between each dose of a multiple dose regimen may vary: for example, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours and/or 24 hours. Thus, in an embodiment, the method disclosed herein comprises administering at least two doses of zinc, or a pharmaceutically acceptable salt thereof, intravascularly to a subject in need thereof. By “at least two doses” means 2, 3, 4, 5, 6, 7, 8, 9, 10, 11 doses, and so on. Thus, in an embodiment, the method disclosed herein comprises administering at least 2 doses, preferably at least 3 doses, preferably at least 4 doses, preferably at least 5 doses, preferably at least 6 doses, preferably at least 7 doses, preferably at least 8 doses, preferably at least 9 doses, and more preferably at least 10 doses of zinc, or a pharmaceutically acceptable salt thereof, to a subject in need thereof. In an embodiment, the period of time between each dose of a multiple dose regimen is about 1 hour, preferably about 2 hours, preferably about 3 hours, preferably about 4 hours, preferably about 5 hours, preferably about 6 hours, preferably about 7 hours, preferably about 8 hours, preferably about 9 hours, preferably about 10 hours, preferably about 11 hours, preferably about 12 hours, preferably about 13 hours, preferably about 14 hours, preferably about 15 hours, preferably about 16 hours, preferably about 17 hours, preferably about 18 hours, preferably about 19 hours, preferably about 20 hours, preferably about 21 hours, preferably about 22 hours, preferably about 23 hours and more preferably about 24 hours. In certain embodiments, the zinc, or the pharmaceutically acceptable salt thereof, is administered to the subject as two separate doses administered within about 36 hours, preferably within about 24 hours, prior to the noxious insult. In an embodiment disclosed herein, the zinc, or the pharmaceutically acceptable salt thereof, is administered to the subject as two separate doses administered at about 24 hours and at about 4 hours prior to the noxious insult.

It has been unexpectedly found that significant advantages in terms of inhibiting the effect of the noxious insult on the organ can be achieved by administering at least two separate dosages of zinc, or of a pharmaceutically acceptable salt thereof. However, in some instances, there may be an upper limit to the number of dosages which are required to achieve that benefit. That is, beyond a certain number of dosages, no further advantage is obtained. Thus, is some embodiments, it will be unnecessary to administer the zinc, or a pharmaceutically acceptable salt thereof, via a multiple dose regimen, as this may run the risk of overdosing Without wishing to be bound by any particular theory or mode of action, it is postulated that the first administration of the zinc, or of a pharmaceutically acceptable salt thereof, ‘primes’ the organ for an improved or optimal response to a second or subsequent dose of zinc, or of a pharmaceutically acceptable salt thereof.

In an embodiment disclosed herein, the method comprises administering to the subject from 2 to 5 doses of zinc, or a pharmaceutically acceptable salt thereof. In another embodiment, the methods disclosed herein comprises administering to the subject from 2 to 5 doses of zinc, or a pharmaceutically acceptable salt thereof, within a 36 hour period prior to the noxious insult.

In certain embodiments, the method disclosed herein involves 2, 3 or 4 separate doses of zinc, or a pharmaceutically acceptable salt thereof, within a 36 hour period prior to the noxious insult. Suitably, there are two separate administrations within any of the time periods previously described.

In other embodiments, the doses of zinc, or a pharmaceutically acceptable salt thereof, within the 36 hour period prior to the noxious insult are administered within a 30 hour, 28 hour, 26 hour or about 24 hour period prior to the noxious insult.

In preferred embodiments, a first dose of zinc, or a pharmaceutically acceptable salt thereof, is administered to the subject between 36 to 12 hours prior to the noxious insult and a second dose is administered between 1 to 10 hours prior to the noxious insult.

In certain embodiments, the first dose is administered between 30 to 18 hours prior to the noxious insult and the second dose is administered between 2 to 6 hours prior to the noxious insult.

The first dose may be administered between 28 to 20 hours prior to the noxious insult and the second dose is administered between 3 to 5 hours prior to the noxious insult, such as at about 24 hours prior to the noxious insult for the first dose and about 4 hours prior to the noxious insult for the second dose.

In another embodiment, therefore, there is provided a method of inhibiting an effect of a noxious insult to an organ of a subject, the method comprising administering zinc, or a pharmaceutically acceptable salt thereof, to a subject in need thereof wherein the zinc, or the pharmaceutically acceptable salt thereof, is administered to the subject intravascularly in an amount that is sufficient to inhibit the effect of the noxious insult and wherein the zinc, or a pharmaceutically acceptable salt thereof, is administered in at least two but less than 5 separate doses within 36 hours prior to the noxious insult. In an embodiment, the method comprises administering the zinc, or the pharmaceutically acceptable salt thereof, to a subject intravenously. In another embodiment, the method comprises administering the zinc, or the pharmaceutically acceptable salt thereof, to a subject intra-arterially.

The number of doses, timing of dosing, time between dosing, and the amount provided in each dose may vary, as previously described.

In certain embodiments, two separate doses are administered. For example, the first dose of zinc, or a pharmaceutically acceptable salt thereof, may be administered to the subject between 36 to 12 hours prior to the noxious insult and the second dose may be administered between 1 to 10 hours prior to the noxious insult.

Each dose of zinc may be administered in an amount that is selected from the group consisting of from about 0.1 mg/kg body weight to about 0.9 mg/kg body weight.

In certain embodiments, the invention comprises the step of selecting a patient in need of treatment for inhibiting an effect of a noxious insult to an organ, such as a kidney, prior to dispensing said treatment. The selection may involve the step of identifying a patient who is to be subjected to an ischaemic event due to medical/therapeutic intervention.

The phrase “pharmaceutically acceptable”, as used herein, refers to molecular forms and compositions that do not produce an adverse, allergic or other untoward reaction when administered to a subject, such as, for example, a human.

In certain embodiments, the zinc, or the pharmaceutically acceptable salt thereof, is formulated with a “pharmaceutically acceptable carrier”. The phrase “pharmaceutically acceptable carrier”, as used herein, means a composition or vehicle, such as a liquid, diluent, excipient or solvent, used to deliver the zinc to the subject. Each carrier should be “acceptable” in the sense of being compatible with zinc, or a pharmaceutically acceptable salt thereof, whilst also being relatively inert: that is, not injurious to the subject. Suitable carriers will be known to persons skilled in the art, illustrative examples of which include solvents, dispersion media, antioxidants, preservatives (e.g., antibacterial agents, antifungal agents), isotonic agents, absorption delaying agents, salts, preservatives, stabilizers, excipients, or combinations thereof. The zinc, or the pharmaceutically acceptable salt thereof, may also be formulated with one or more other active agents or ingredients.

As noted elsewhere herein, the phrase “therapeutically effective amount” refers to an amount of zinc, or a pharmaceutically acceptable salt thereof, that is effective to reduce or inhibit an effect of a noxious insult to an organ, such as a kidney. A therapeutically effective amount can be readily determined by conducting a clinical trial or metabolic studies in experimental animals, such as sheep, to determine an optimum range of doses that will provide the desired outcome. Before use in a clinical setting, it can be beneficial to conduct confirmatory studies in an animal model, typically a widely accepted animal model of the particular disease to be treated. Typical animal models for use in certain embodiments are rodent, murine and ovine models, which are economical to use and because the results gained are indicative of clinical value (i.e., in human subjects).

### Subject

The term “subject” as used herein refers to an animal, in particular a mammal and more particularly a primate including a lower primate and even more particularly, a human who can benefit from the methods disclosed herein. A subject, regardless of whether a human or non-human animal or embryo, may be referred to as an individual, subject, animal, patient, host or recipient. The present invention has both human and veterinary applications. For convenience, an “animal” specifically includes domestic and livestock animals such as dogs, cats, cattle, horses, sheep, pigs, camelids, goats and donkeys. With respect to horses, these include horses used in the racing industry as well as those used recreationally or in the livestock industry. Examples of laboratory test animals include mice, rats, rabbits, guinea pigs and hamsters. Rabbits and rodent animals, such as rats and mice, provide a convenient test system or animal model as do primates and lower primates. In an embodiment disclosed herein, the subject is human.

In another embodiment disclosed herein, the subject has renal cancer.

In another embodiment disclosed herein, the subject has or is at risk of a myocardial infarction.

In a second aspect, there is provided zinc, or a pharmaceutically acceptable salt thereof, for use in inhibiting an effect of a noxious insult to an organ of a subject, wherein the zinc, or the pharmaceutically acceptable salt thereof, is administered intravascularly to a subject in need thereof in an amount that is sufficient to inhibit the effect of the noxious insult.

In a third aspect, there is provided use of zinc, or a pharmaceutically acceptable salt thereof, in the manufacture of a medicament for inhibiting an effect of a noxious insult to an organ of a subject, wherein the zinc, or the pharmaceutically acceptable salt thereof, is formulated for intravascular administration to a subject in need thereof in an amount that is sufficient to inhibit the effect of the noxious insult.

The use of the second and third aspects may be performed in accordance with those embodiments described for the first aspect.

Preferred embodiments of this invention are described herein, including the best mode known to the inventors for carrying out the invention. Variations of those preferred embodiments may become apparent to those of ordinary skill in the art upon reading the foregoing description. It is expected that skilled artisans will employ such variations as appropriate and it is considered within the scope and spirit of the present invention for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The invention will now be described by, but it is in no way limited to, the following Examples.

### EXAMPLES

### Example 1: Intravenous Zinc Administration Inhibits Ischemia-Reperfusion Injury in Sheep Kidneys

### Materials and Methods

### A. Zinc Administration

Intravenous injections of pharmaceutical grade zinc chloride (ZnCl₂) solution was administered to sheep [n=4] via an implanted jugular vein catheter at a dose ranging from 0.1 to 2 mg/kg of body weight. The sheep were administered the ZnCl₂ solution at 24 hours and 4 hours prior to surgery. Following the 4 hour injection, day 0 blood samples were taken. Preliminary results showed that sheep tolerated these doses of ZnCl without any adverse effects.

Controls (n=4) received a unilateral nephrectomy and were administered saline at 24 hours and 4 hours prior to surgery.

Another group of sheep (n=1) received the Flavonoid based antioxidant (3′,4′ dihydroxyflavonol) at 6.6 mg/kg of body weight 1 hours prior to surgery.

### B. Anaesthesia

Sheep were anaesthetised prior to surgery with intravenous sodium thiopentone (15 mg/kg) for endotracheal intubation (cuffed tube size 10). Maintenance of anaesthesia was carried out by means of oxygen/air/isoflurane (1.5-2.0%). Fractional inspired oxygen was altered to maintain PaO₂ at approximately 100 mmHg, and ventilation was controlled to maintain PaCO₂ at approximately 40 mmHg.

### C. Surgical Procedure and Sample Analysis

Following general anaesthesia, the right kidney was removed. A flow probe (4 mm) was implanted into the left kidney and the renal hilum (arteries and veins) supplying blood to the kidney were clamped using a vascular clamp for 60 minutes. Following 60 minutes of ischaemia, the vascular clamp was removed from the left kidney and the blood supply restored. General anaesthesia was then reversed and the sheep were allowed to recover. Renal blood flow was measured, blood samples were taken daily from the sheep over a 1-week period and the level of urea and creatinine was determined. Blood samples were collected into lithium heparin tubes (Microtainer, Becton-Dickinson). Blood samples were centrifuged and aliquots stored at −20° C. till analysis for creatinine and urea by the Clinical Trials Department, Austin Pathology, Heidelberg, Australia. Creatinine and urea in the blood serum samples were measured on a fully automated Roche Cobas 8000 c702 analyser. The kinetic colorimetric assay for creatinine determination is based on the Jaffé method. The kinetic colorimetric assay for urea is based on hydrolyzation of Urea to ammonium catalysed by urease followed by reaction of ammonium with 2-oxoglutarate in the presence of glutamate dehydrogenase and the coenzyme NADH. The rate of decrease in the NADH concentration is directly proportional to the urea concentration in the specimen and is measured photometrically.

### Results

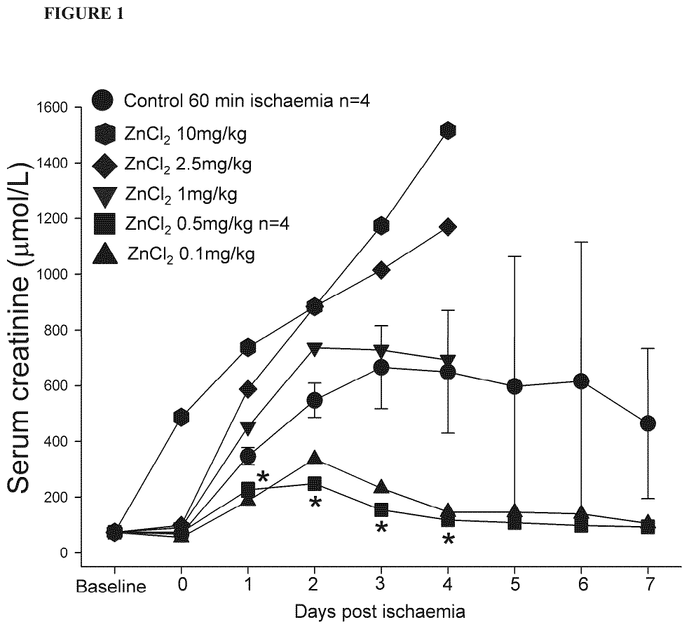

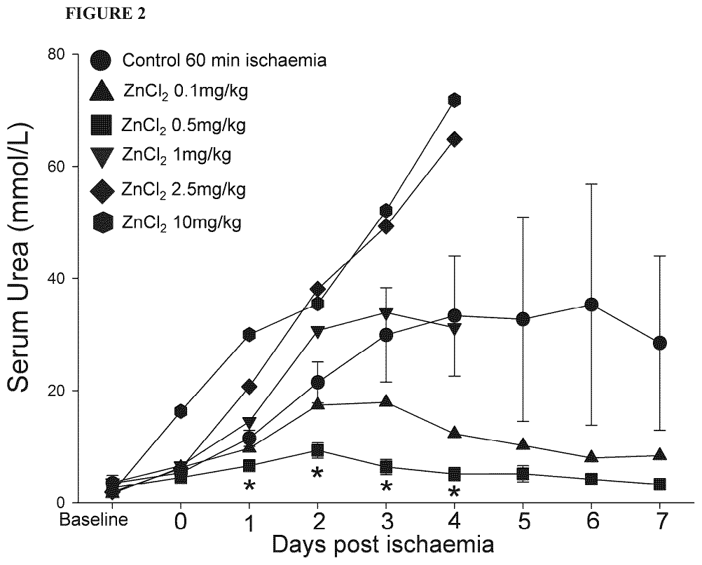

ZnCl at 0.1 and 0.5 mg/kg of body weight inhibited ischaemia-reperfusion injury in the sheep kidney, as evidenced by a marked reduction in serum creatinine (FIG. 1) and serum urea (FIG. 2) levels when compared to controls. The reduction in serum creatinine levels reached statistical significance on days 1-4 following ischaemia-reperfusion injury and the reduction in serum urea levels reached statistical significance on days 1, 3 and 4 following ischaemia-reperfusion injury (* p<0.05).

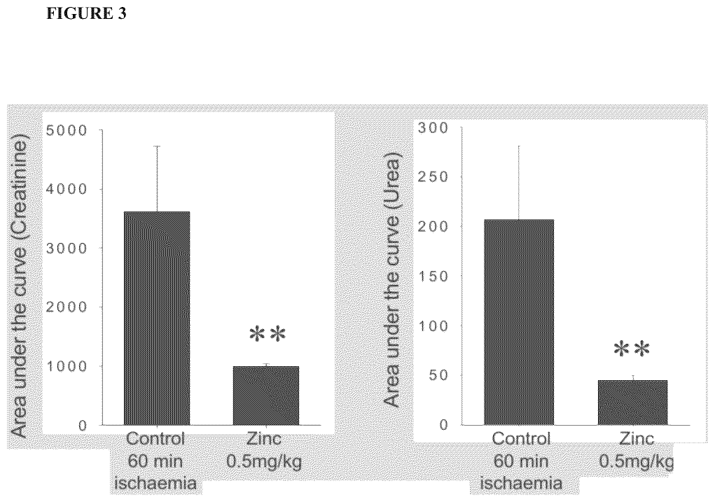

Ischaemic burden, as evidenced by the AUC (area under the curve) values for serum creatinine and serum urea, was reduced by nearly 70% with 0.5 mg/kg zinc when compared to controls animals (FIG. 3: ** p<0.05).

ZnCl at 1.0, 2.5 and 10.0 mg/kg of body weight failed to inhibit ischaemia-reperfusion injury, as measured by serum creatinine and urea levels when compared to controls. Moreover, the data also shows that ZnCl at 2.5 and 10.0 mg/kg of body weight appeared to worsen the adverse effects of ischaemia-reperfusion in sheep kidneys, as evidenced by a trend towards higher serum creatinine and urea levels when compared to controls.

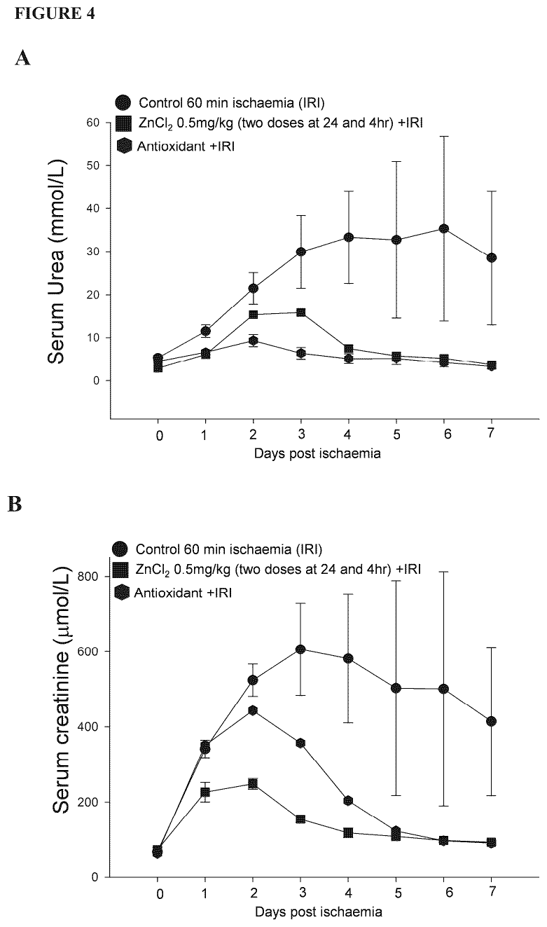

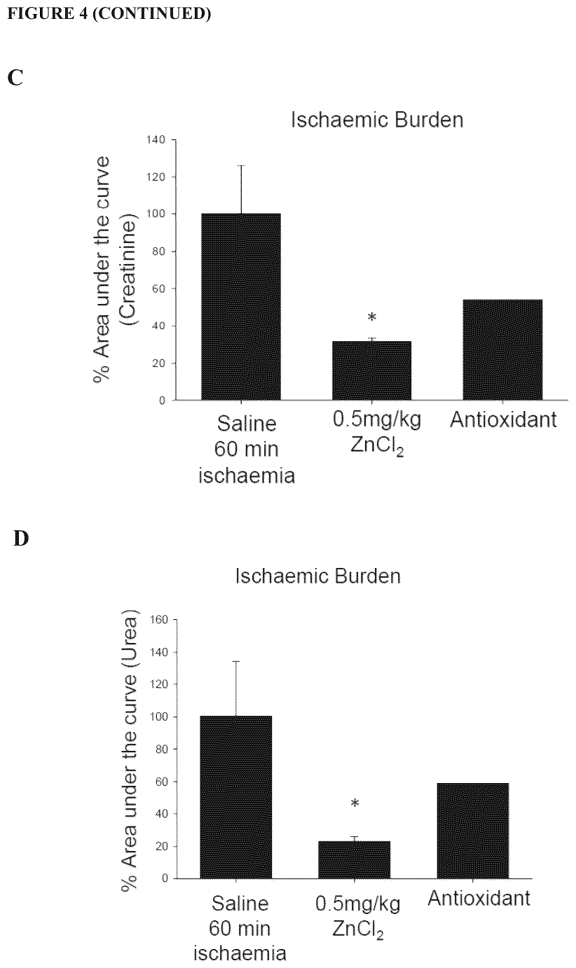

The degree of inhibition of ischemia-reperfusion injury, as evidenced by a reduction in serum creatinine levels, was greater than the degree of inhibition attributed to the antioxidant (FIGS. 4A and 4B). Ischaemic burden, as evidenced by the AUC (area under the curve) values for serum creatinine and serum urea, was reduced by nearly 70% with 0.5 mg/kg zinc when compared to only 50% decrease in antioxidant treated animals (FIGS. 4B and 4C: ** p<0.05).

### Example 2: Zinc Preconditioning Protects Kidneys from Ischaemia-Reperfusion Injury Partially Via Upregulation of Hypoxia Inducible Factor 1-Alpha (HIF-1α)

### A. Human Study

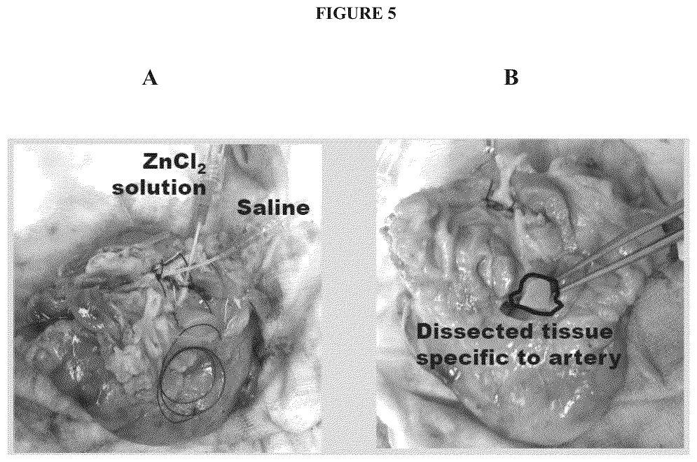

After separation from the cancerous portion, segmental arteries in a normal human kidney were dissected out, perfused with 50 μM ZnCl2 or saline and then tissue specific to that artery was harvested for HIF-1α evaluation. FIG. 5 shows the perfusion of ZnCl₂ (50 μM) into a human kidney via the segmental renal artery (A) and the renal tissue that was subsequently removed for analysis (B). The blood supply to the excised tissue was provided by the renal artery into which the ZnCl₂ solution was administered. In an initial study a 25% increase in HIF-1α expression in normal human kidney perfused with Zn compared to saline perfused tissue was observed.

### B. In Vitro Study

Zn stimulated HIF1α in HK-2 cells. Immortalized human kidney tubular cells, HK-2, were treated with 50 μM ZnCl₂ and changes to HIF1α protein expression were measured by Western blot analysis.

Zn stimulated HIF1α in ACHN cells. Human renal carcinoma cells (ACHN) were treated with 50 μM ZnCl₂ and changes to HIF1α protein expression were measured by Western blot analysis.

### Results

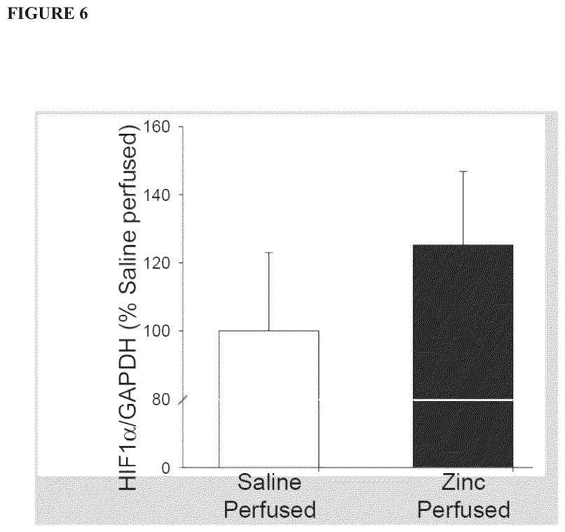

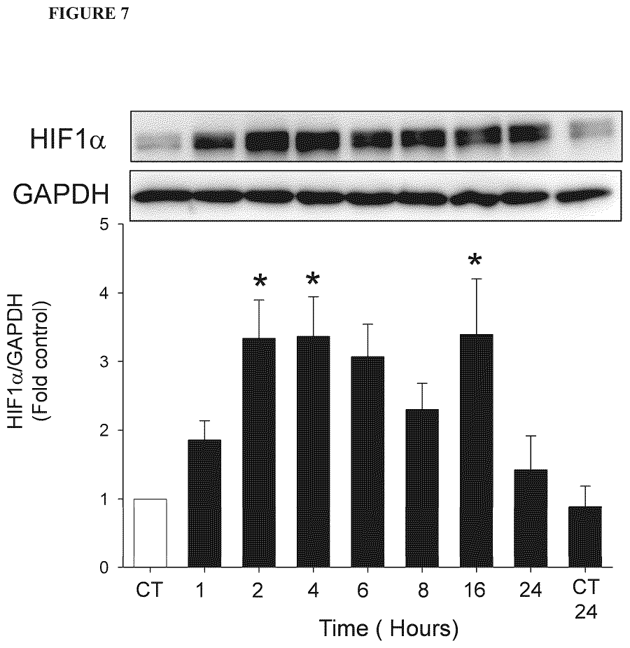

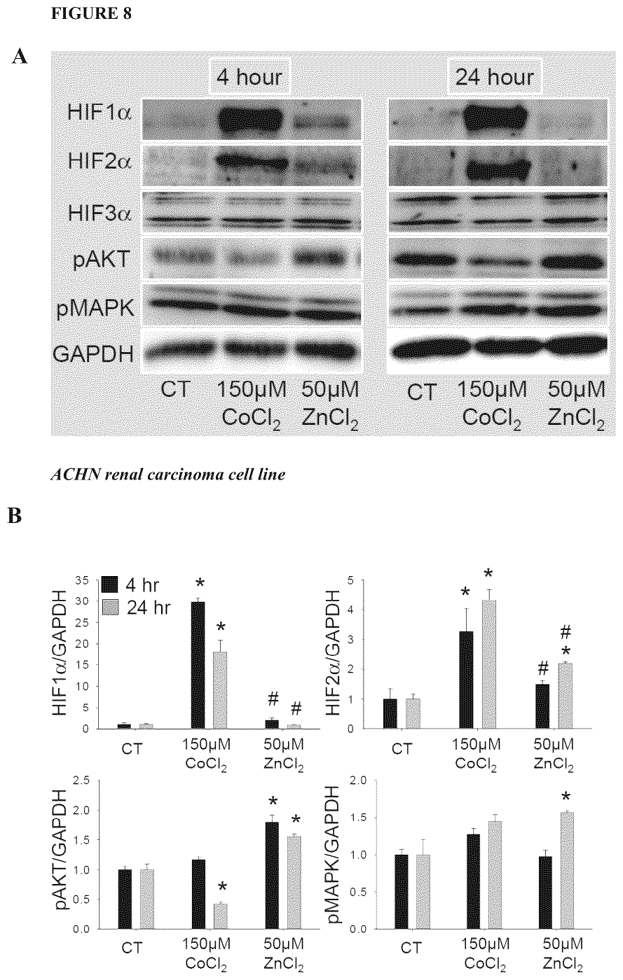

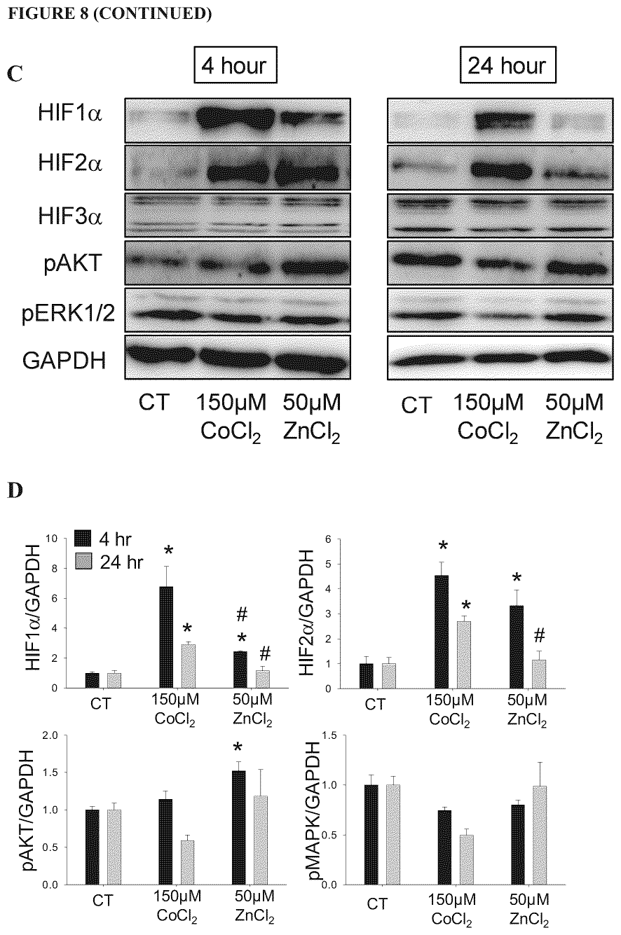

Zinc chloride preconditioning enhanced HIF-1α protein expression in human kidney tissue (see FIG. 6). Zinc chloride also enhanced HIF-1α protein expression in immortalised human proximal renal tubule cells (HK2: FIG. 7) and in human renal carcinoma cells (ACHN: FIGS. 8A and 8B) at least 4 hours following exposure.

HIF-1α modulates a cell's response to hypoxia and its upregulation has been shown to induce the expression of several genes involved in adaptation to decreased oxygen availability. HIF-1α is a heterodimeric protein comprising an oxygen-regulated alpha subunit and a constitutive expressed beta subunit. Alpha subunits are degraded during normoxia, primarily through a proteasome-dependent pathway after hydroxylation of two proline residues by prolyl-hydroxilases (PHDs). During hypoxia, PHDs are inhibited and HIF-1α subunit accumulates, where it dimerizes with HIF-1ß to drive expression of HIF target genes, such as those involved in angiogenesis and tissue repair (e.g., vascular endothelial growth factor (VEGF), erythropoietin (EPO), prolilhydroxilases (PHDs) genes).

A study by Elisa Conde and her colleagues (PlosOne, March 2012, Vol. 7(3):e33258) found that HIF-1α is stabilized in proximal tubule cells of rat kidneys during ischaemia and unexpectedly in late reperfusion, when oxygen tension is normal. Moreover, in vitro interference of HIF-1α expression promoted cell death and in vivo interference exacerbated tissue damage and renal dysfunction following ischaemia-reperfusion injury. The authors also reported that HIF-1α was expressed only in proximal tubules of post-transplant human biopsies that exhibited normal renal structure, with a significant negative correlation with acute tubular necrosis. These data are consistent with the present data, which show that zinc preconditioning enhances HIF-1α protein expression in rat kidneys and that zinc stimulates HIF-1α expression in human kidney tubular renal cells (FIG. 7) and human renal carcinoma cells (FIG. 8). Without being bound by theory or by a particular mode of application, these data suggests that zinc preconditioning protects kidneys from ischaemia-reperfusion injury, at least in part, via upregulation of HIF-1α. Furthermore, zinc stimulated HIF-2α but not HIF-3a in both ACHN and HK2 cells. Treatment with ZnCl₂ for 4 hours stimulated phosphorylation of AKT in both ACHN (FIGS. 8A and 8B) and HK-2 cells (FIGS. 8C and 8D) but activated phosphorylation of ERK1/2 only in ACHN cells at 24 hours.

### Example 3: Effect of Multiple Dosing with Zinc

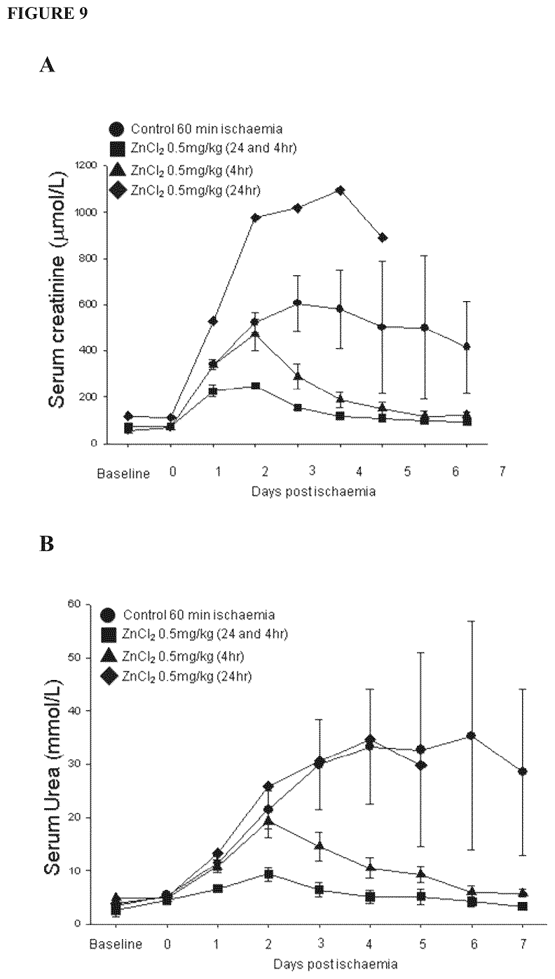

As 0.5 mg/kg had been established as an effective dose to inhibit renal ischemia-reperfusion injury, the dosing experiments above were repeated to investigate the effect of multiple dosing. Sheep were subjected to uninephrectomy and 60 min of renal ischaemia followed by reperfusion. Serum creatinine and serum urea levels were measured before Zn pre-treatment (baseline), before ischaemia (Day 0) and for 7 days after reperfusion. Sheep were preconditioned with a single dose of 0.5 mg/kg ZnCl₂ either at 24 hours or 4 hours only, or with a double dose of 0.5 mg/kg ZnCl₂ at 24 and 4 hours prior to 60 min ischaemia. Data are expressed as mean±SEM values (n=4, except 24 hr only group, n=1). FIG. 9 shows that ZnCl₂ administered as two doses (at 0.5 mg/kg body weight) at 24 hours and at 4 hours prior to ischaemia significantly reduced the creatinine rise over time compared to a single dose given only at either 24 hours or 4 hours prior to ischaemia (p<0.05). It can be seen from these data that the dual dosing approach gave significantly better outcomes when compared to a single dose of 0.5 mg/kg ZnCl₂ given at either 4 hours or 24 hours pre-ischemia.

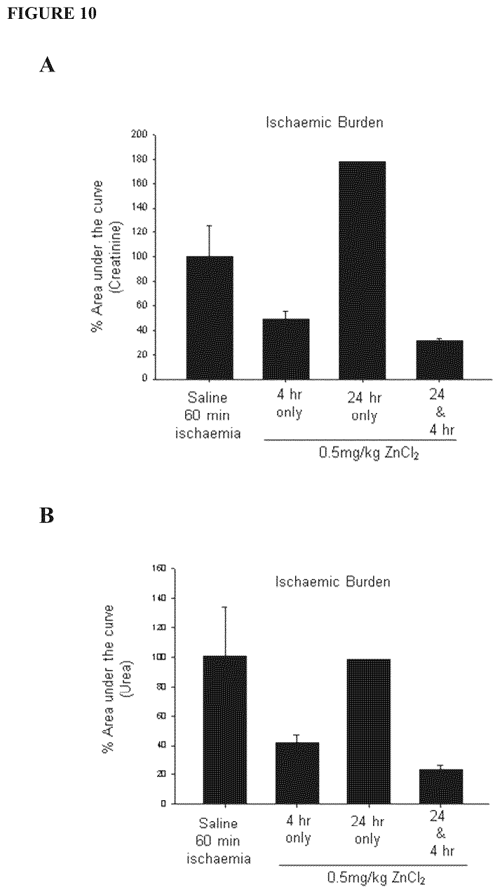

Ischaemic burden, as evidenced by the AUC (area under the curve) values for serum creatinine and serum urea, was reduced significantly with 0.5 mg/kg zinc dosed at 24 hours and then at 4 hours pre-ischaemia when compared to controls animals (FIG. 10) and individual 24 hour and 4 hour dosing.

### Example 4: Zinc Preconditioning Protects Myocardial Tissue from Ischaemia-Reperfusion Injury

Myocardial ischaemia-reperfusion was performed in adult sheep (30-50 kg). Sheep were preconditioned with dual doses of 0.5 mg/kg ZnCl₂ at 24 hours and 4 hours prior to myocardial ischaemia. Following a left thoracotomy and opening of the pericardium, the second branch of the left anterior descending coronary artery (D2) was isolated and a vascular snare was placed proximal to a transit-time flow probe (2 mm). Following stabilization, the experimental protocol consisted of 30 min baseline recording of systemic hemodynamics and coronary flow, followed by 1 hour ischemia and 3 hour reperfusion. The area of myocardium at risk of infarction and infarct size were delineated by Evan's blue and triphenyltetrazolium chloride (TTC) staining, respectively, as previously described (Wang et al., 2004. Br. J. Pharmacol. 142, 443-452). Briefly, after 3 hour reperfusion, the D2 artery was re-occluded with the vascular snare. Immediately prior to arresting the heart with an intravenous injection of pentobarbitone (100 mg/kg: Virbac, Australia), Evan's blue dye (1.5% in 60 ml saline, Sigma, Australia) was injected into the left atrial catheter to define the ischaemic myocardium: i.e., the area at risk of infarction. The heart was rapidly excised and the left ventricle was sliced into six, 1 cm thick transverse rings which were photographed and the unstained areas traced onto transparencies. The rings were incubated in 0.1 M sodium phosphate buffer containing 1% TTC (Sigma, Australia) for 20 min at 37° ° C., pH 7.4, re-photographed and traced onto transparencies with the infarcted area outlined. The area of myocardium at risk of infarction and the infarct size were measured by computerized planimetry (MCID-M 2, Imaging Research Inc., Canada). The myocardium-at-risk was expressed as a percentage of total left ventricular volume and infarct size was expressed as a percentage of the area of myocardium at risk.

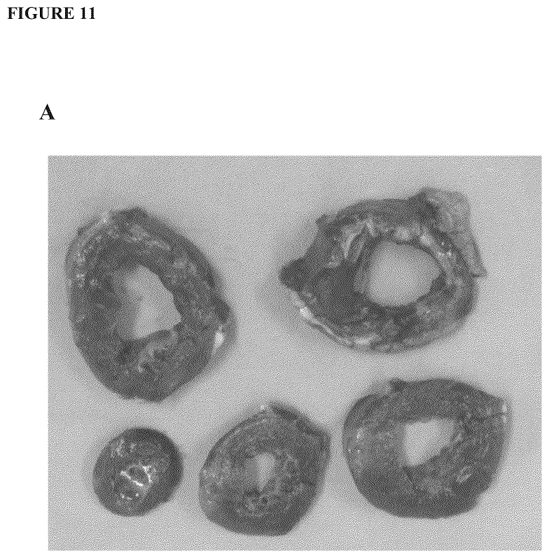

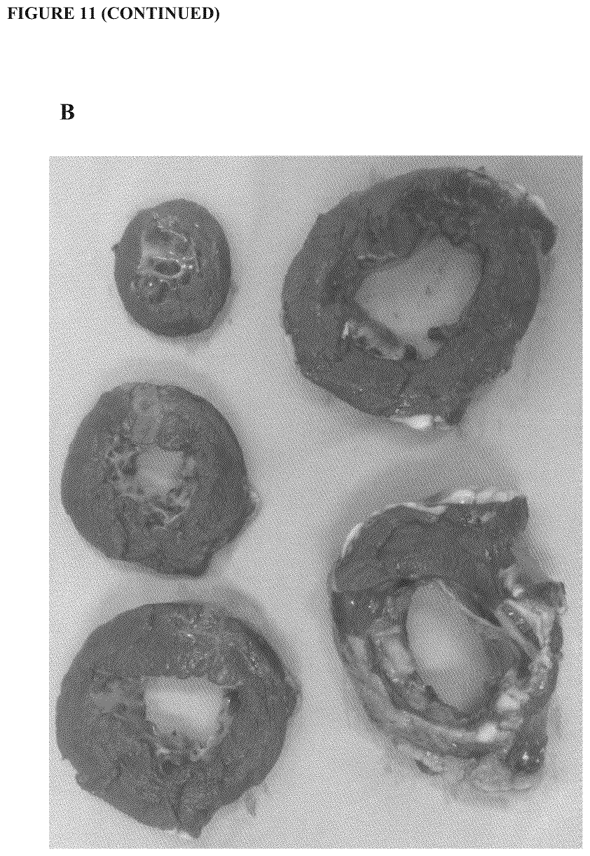

FIG. 11 shows two sets of photos of sections of sheep heart, pre- and post-TCC staining, from this study. The first photo (FIG. 11A) shows transverse rings stained with Evans blue, prior to any TCC staining. It highlights the areas at risk of ischaemia, and represents the territory supplied by the vasculature vessel which was clamped in the experiment. The second photo (FIG. 11B) shows transverse rings after contact with TCC buffer, whereby the infarcted tissue turns white. It is apparent from FIG. 11B that there was very little white staining to be seen, which is indicative of a significant reduction of damage from ischaemia or ischaemia-reperfusion as a result of zinc pre-conditioning. When the infarct size was expressed as a percentage of the area of myocardium at risk (as previously described in Thomas et al, 2011, European Journal of Pharmacology 658: 160-167), the infarct size was 80±3% of the area of myocardium in control (non zinc-treated) animals, whereas the zinc-treated animal had no histological signs of an infarct in the area of myocardium at risk.

1. A method of inhibiting an effect of a noxious insult to an organ of a subject, the method comprising administering zinc, or a pharmaceutically acceptable salt thereof, to a subject in need thereof
wherein the zinc, or the pharmaceutically acceptable salt thereof, is administered to the subject intravascularly in an amount that is less than or equal to 1 mg/kg body weight,
wherein the noxious insult is ischaemia and/or ischaemia-reperfusion, and
wherein the zinc, or the pharmaceutically acceptable salt thereof, is administered to the subject prior to the noxious insult.

2-5. (canceled)

6. The method of claim 1, wherein the noxious insult results from a surgical procedure.

7. The method of claim 1, wherein the ischaemia noxious insult occurs during resection of a tumour.

8. The method of claim 1, wherein the noxious insult is to a donor organ during an organ transplant.

9. (canceled)

10. The method of claim 1, wherein the zinc, or the pharmaceutically acceptable salt thereof, is administered via perfusion to the organ.

11. The method of claim 1, wherein the zinc, or the pharmaceutically acceptable salt thereof, is administered via a peripheral or central vein.

12. (canceled)

13. The method of claim 1, wherein the zinc, or the pharmaceutically acceptable salt thereof, is administered in a solution.

14. The method of claim 13, wherein the solution comprises zinc chloride.

15. The method of claim 1, wherein the zinc, or the pharmaceutically acceptable salt thereof, is administered intravenously and/or intra-arterially.

16. The method of claim 1, wherein the zinc, or the pharmaceutically acceptable salt thereof, is administered to the subject within 36 hours prior to the noxious insult.

17. The method of claim 1, wherein the zinc, or the pharmaceutically acceptable salt thereof, is administered to the subject within 4 hours prior to the noxious insult.

18. The method of claim 1, wherein the zinc, or the pharmaceutically acceptable salt thereof, is administered to the subject in multiple doses within 36 hours prior to the noxious insult.

19. The method of claim 18, wherein the zinc, or the pharmaceutically acceptable salt thereof, is administered to the subject in two separate doses.

20. The method of claim 19, wherein a first dose of zinc, or a pharmaceutically acceptable salt thereof, is administered to the subject between 36 to 12 hours prior to the noxious insult and a second dose of zinc, or a pharmaceutically acceptable salt thereof, is administered to the subject between 1 to 10 hours prior to the noxious insult.

21. The method of claim 20, wherein both the first dose and the second dose are between 0.1 mg/kg and 0.9 mg/kg.

22. The method of claim 1, wherein the subject is a human.

23. The method of claim 1, wherein the organ of the subject is selected from the group consisting of kidney, liver, heart and brain.

24. The method of claim 23, wherein the subject has renal cancer.

25-29. (canceled)
